(19) United States
(12) Patent Application Publication
(10) Pub. No.: US 2018/0243031 A1
(43) Pub. Date: Aug. 30, 2018

(54) Ablation Catheter with Sensor System for Detecting the Ablation Success

(71) Applicant: Vimecon GmbH, Herzogenrath (DE)

(72) Inventor: Kai Ulf Markus, Eschweiler (DE)

(21) Appl. No.: 15/757,161

(22) PCT Filed: Aug. 30, 2016
(86) PCT No.: PCT/EP2016/070406

§ 371 Date: Apr. 20, 2018

(30) Foreign Application Priority Data
Sep. 3, 2015 (DE) 10 2015 216 891.3

(51) Int. Cl. A61B 18/24

(52) U.S. Cl. CPC A61B 18/24; A61B 2017/00061

(57) ABSTRACT

An ablation catheter for ablation of biological tissue, including at least one optical fiber for transporting laser light along the ablation catheter and at least one coupling-out region for decoupling the laser light transported by the optical fiber from the catheter. A sensor system that continuously detects parameters, from which ablation success can be determined.

The invention relates to an ablation catheter for ablation of biological tissue. The ablation of biological tissue is typically performed using laser light. One application is the ablation of the myocardial muscle for stopping a deficient pulse transmission upon excitation of the myocardial muscle. Using an optical fiber, the laser light is transported along the ablation catheter into the ablation zone. In the ablation zone, i.e. the site, where the ablation of tissue is to be performed, the laser light is decoupled from the catheter via a coupling-out portion into the tissue surrounding the catheter or the tissue in contact with the catheter.

The power induced by the laser into the tissue to be ablated is chosen such that the tissue in the region of deficient pulse transmission is heated and the pulse transmission is thereby disturbed or stopped.

In ablating biological tissue, the selection of the laser power and the well-aimed ablation are of particular importance. Healthy tissue is not to be affected.

Basically, it is a difficulty in ablating biological tissue using an ablation catheter to detect the ablation success already during ablation.

Therefore, it is an object of the invention to provide an ablation catheter with which the ablation success can be detected.

The ablation catheter of the present invention is defined by the features of claim 1. According thereto, the ablation catheter is provided with a sensor system adapted to continuously detect parameters from which the ablation success can be determined. The parameters may e.g. be the color of the ablated tissue, the heat in the ablation zone of the catheter or the expansion of the catheter. Based on the color of the ablated tissue, conclusions may be drawn on the degree of heating or the temperature of the ablated tissue. When heated correspondingly, biological tissue changes its color from red to black via grey. From the heat in the ablation zone of the catheter, it is possible to conclude on the tissue in contact with the catheter in the ablation zone. Finally, the expansion of the catheter allows to draw conclusions on the temperature of the catheter and thus also on the temperature of the tissue in contact with the catheter.

Basically, two principles are conceivable for the sensor system of the invention:

On the one hand, at least one coupling-in region may be formed for coupling an electromagnetic wave into the catheter, so that the wave is transmitted in the proximal direction through the catheter to an evaluation unit arranged outside the catheter. Here, the wave coupled-in can be transmitted through the optical fiber of the catheter or through a waveguide extending through the catheter in parallel with the optical fiber, which waveguide may e.g. be a light waveguide or a waveguide for the transmission of another form of electromagnetic wave. The waveguide may in particular be an electric connection line for the transmission of an electric signal. The coupling-in region may be part of the coupling-out region. In this case, the electromagnetic wave is coupled e.g. into the optical fiber of the catheter through at least a part of the coupling-out region. As an alternative or in addition, at least one coupling-in region may be provided adjacent the coupling-out region.

On the other hand, it is conceivable that at least one sensor is provided at or adjacent the coupling-out region, wherein a connecting line extending through the catheter connects the sensor to an evaluation unit arranged outside the catheter. This connection may be an electric connection for transmitting an electric signal generated by the sensor to the evaluation unit. In this variant, the sensor may be a photo sensor or a heat sensor.

In the variant using the coupling-in region, it is possible e.g. to couple light into the catheter and to transmit the same to the evaluation unit in the proximal direction. Here, the evaluation unit is configured to recognize the wavelength of the light coupled in as an indication of an ablation success based on the color of the ablated tissue. As an alternative or in addition, thermal radiation coupled into the coupling-in region can be transmitted in the proximal direction through the catheter to the evaluation unit, wherein the evaluation unit is configured to detect thermal or infrared radiation as an indication of the heat in the ablation zone.

The color of the ablated tissue may also be detected using a photo sensor as a part of the sensor system. Using a photo sensor, it is also possible to detect the heat (infrared radiation) in the ablation zone of the catheter. The expansion of the catheter may be detected electrically, by first generating a stationary electromagnetic wave in the catheter and measuring the phase shift of the stationary electromagnetic wave, so as to determine the expansion of the catheter from the phase shift. The connection between the catheter temperature and the catheter expansion is known and is typically linear. The heat in the ablation zone of the catheter may also be detected in a general manner using a heat sensor.

In a preferred embodiment the sensor system is configured for a position-resolved detection of the parameters. The position-resolved detection may e.g. be determined based on the phase shift of the electromagnetic wave formed in the catheter. As an alternative or in addition, in the case of a photo sensor, it is conceivable to use a material that is translucent depending on the temperature.

It is a basic advantage of the invention that the detection of the parameters by the sensor system is performed in a continuous manner so as to be able to continuously detect and monitor the ablation success during ablation. The evaluation unit is arranged outside the catheter. In the variant with a coupling-in region and a wave guide, also the sensors are arranged outside the catheter and sensor in the catheter are not required.

The following is a detailed explanation of embodiments of the invention with reference to the drawings. In the Figures:

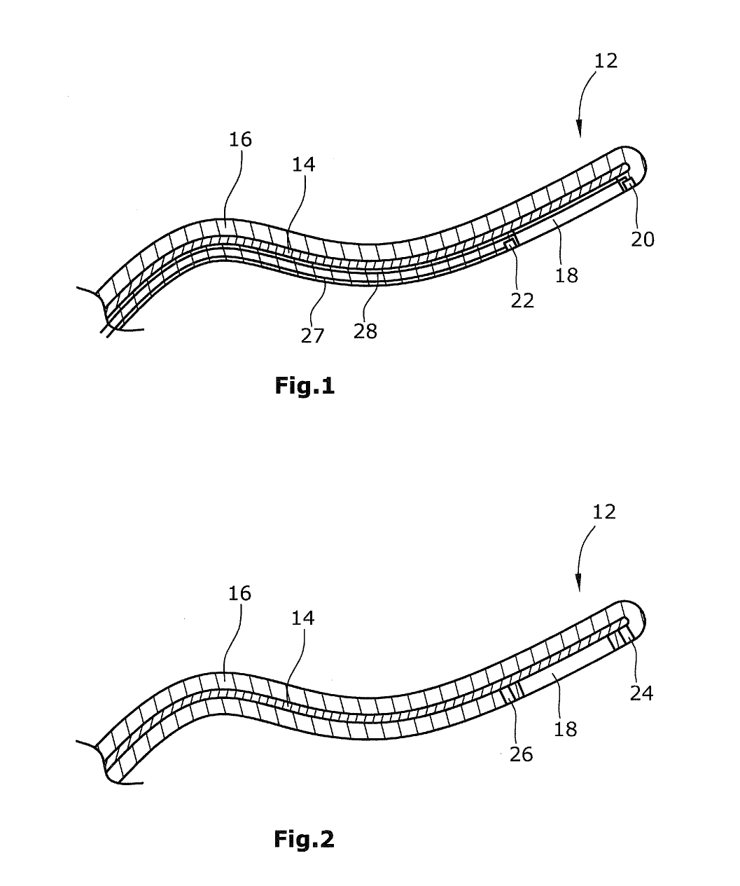

FIG. 1 is a longitudinal section through a distal end portion of the ablation catheter of the first embodiment, and

FIG. 2 shows the longitudinal section of FIG. 1 according to the second embodiment.

In a manner known per se, the ablation catheter 12 comprises an optical light guide fiber 14 inside the catheter 12. The light guide fiber 14 is configured for conveying laser light of the necessary wavelength and power. The light guide fiber 14 is surrounded by at least one catheter sheath 16. In the distal end region, the ablation catheter 12 is provided, in a manner also known per se, with a coupling-out region 18 through which the laser light transported by the optical fiber 14 is decoupled from the catheter 12. Coupling out the laser light in the coupling-out region 18 is typically performed in a directed manner only in the region of a partial circumference of the catheter sheath, so as to enable a well-aimed ablation.

In the variant of FIG. 1 sensors 20, 22 are provided distally and proximally of the coupling-out region 18. The sensors 20, 22 are each embedded in the material of the catheter sheath 16. The sensors 20, 22 may be photo sensors, heat sensors and/or electromagnetic sensors for detecting the phase of the electromagnetic wave formed in the catheter 12. Via an electric connection line 27, 28, the sensors 20, 22 are each connected to an evaluation unit arranged outside the catheter, the evaluation unit detecting and evaluating the electric signals generated by the sensors 20, 22. The electric connection lines 27, 28 are embedded in the material of the catheter sheath 15 and extend parallel to the optical fiber 14.

In the embodiment of FIG. 2, a respective coupling-in region 24, 26 is provided distally and proximally of the coupling-out region 18 adjacent the coupling-out region. Via the coupling-in regions 24, 26 an electromagnetic wave, e.g. a light wave or thermal radiation, is coupled into the catheter 12 and is transmitted in the proximal direction along the catheter to an evaluation unit not illustrated in the Figures and arranged outside the catheter 12. The evaluation unit is provided with suitable sensors which detect the wave coupled in and generate an electric or electronic signal. In the variant in FIG. 2, the coupling-in regions 24, 26 are connected to the optical fiber 14 such that a wave from outside the catheter 12 is coupled from the ablation zone into the optical fiber 14 so as to be transmitted in the proximal direction through the fiber 14 to the evaluation unit.

As an alternative, a variant is conceivable that is not illustrated in the Figures, wherein the coupling-in regions 24, 26 are connected to a separate waveguide within the catheter 12, extending parallel to the optical fiber 14, so as to transport the wave coupled in through the waveguide to the evaluation unit arranged outside the catheter.

The wave coupled in may be light, thermal radiation or another form of an electromagnetic wave.

Moreover, it is conceivable as an alternative or in addition that the electromagnetic wave is coupled into the optical fiber and/or a waveguide extending in parallel with the optical fiber via at least a part of the coupling-out region 18. Thus, in this variant, at least a part of the coupling-out region 18 is a coupling-in region.

The invention particularly allows for the first time to detect parameters that make it possible to obtain information about the ablation success already during ablation.

1. A system comprising:
an ablation catheter for ablation of biological tissue, comprising:
at least one optical fiber for transporting laser light along the ablation catheter, and
at least one coupling-out region for decoupling the laser light transported by the optical fiber from the ablation catheter; and
a sensor system configured to continuously detect parameters, from which an ablation success can be determined.

2. The system of claim 1, wherein an electromagnetic wave is coupled into the optical fiber through the coupling-out region or through at least one coupling-in region arranged adjacent the coupling-out region, and the optical fiber is connected to sensors for detecting the coupled-in wave outside the ablation catheter.

3. The system of claim 1, wherein the sensor system comprises at least one coupling-in region arranged adjacent the coupling-out region for coupling an electromagnetic wave into the ablation catheter.

4. The system of claim 2, further comprising a waveguide extending through the catheter in parallel with the optical fiber, wherein the coupling-in region is connected to the waveguide extending through the ablation catheter in parallel with the optical fiber, said waveguide being connected outside the ablation catheter to sensors for detecting the wave coupled into the waveguide.

5. The system of claim 1, wherein the sensor system comprises at least one sensor arranged adjacent the coupling-out region, wherein the at least one sensor is connected to an evaluation unit arranged outside the ablation catheter via a connection line extending through the ablation catheter.

6. The system of claim 5, wherein the sensor system comprises at least one photo sensor for detecting a color of ablated tissue.

7. The system of claim 5, wherein the sensor system comprises at least one thermal sensor for detecting heat in an ablation zone.

8. The system of claim 1, wherein the sensor system is configured to detect expansion of the ablation catheter, and to determine heat of the ablation catheter based on the expansion.

9. The system of claim 8, wherein the sensor system is configured to detect the expansion based on a phase shift of a stationary electromagnetic wave formed in the ablation catheter.

10. The system of claim 1, wherein the sensor system is configured for a position-resolved detection of the parameters.

11. The system of claim 10, wherein the position-resolved detection of the parameters is performed based on a phase shift of an electromagnetic wave.

12. The system of claim 10, wherein the ablation catheter comprises a material that is translucent depending on a temperature of the material.
